(19) United States
(12) Patent Application Publication
(10) Pub. No.: US 2003/0125242 A1
(43) Pub. Date: Jul. 3, 2003

(54) Polypeptides comprising multimers of nuclear localization signals or of protein transduction domains and their use for transferring molecules into cells

(76) Inventors: Joseph Rosenecker, Munchen (DE); Wolfgang Ritter, Munchen (DE); Carsten Martin Rudolph, Munchen (DE); Christian Plank, Seefeld (DE)

Correspondence Address: FISH & NEAVE, 1251 AVENUE OF THE AMERICAS, 50TH FLOOR, NEW YORK, NY, 10020-1105, US

(21) Appl. No.: 10/156,570

(22) Filed: May 24, 2002

Related U.S. Application Data
(63) Continuation of application No. PCT/EP00/11690, filed on Nov. 23, 2000.

(30) Foreign Application Priority Data
Nov. 24, 1999 (EP) 99 12 3423.8

(51) Int. Cl. A61K 48/00; A61K 38/08; A61K 38/10; C07K 9/00; C12N 15/85

(52) U.S. Cl. 514/8; 514/12; 514/14; 514/15; 514/16; 530/322; 530/326; 530/327; 530/328; 435/455

(57) ABSTRACT

Described are polypeptides comprising at least two peptide monomers comprising a nuclear localization sequence or a protein transduction domain and their use for transferring molecules into eukaryotic cells, as well as pharmaceutical compositions comprising the described polypeptides and processes for transferring molecules into eukaryotic cells.

The present invention relates to polypeptides which comprise at least two peptide monomers comprising a nuclear localization sequence or a protein transduction domain and their use for transferring molecules, in particular nucleic acid molecules, into eukaryotic cells. The present invention also relates to processes for transferring molecules into eucaryotic cells by using the described polypeptides and to pharmaceutical compositions comprising the polypeptides.

The transport of exogenous polynucleotides into the cytoplasm and the cell nucleus of eukaryotic cells is crucial for the efficiency of gene therapeutical approaches. Most delivery mechanisms used to date involve viral vectors, especially adeno- and retroviral delivery systems. However, also non-viral delivery systems have been developed which are based, e.g., on receptor-mediated mechanisms, on polymer-mediated transfection such as polyamidoamine, dentritic polymer, polyethylene imine or polypropylene imine, polylysine or on lipid-mediated transfection. But there remain major problems like the low rate of transfected cells or the cell toxicity of the vectors. Transfecting a cell with DNA/vector complexes is subject to a number of barriers. Former studies mostly dealt with overcoming the outer cell membrane. A lot of strategies were developed resulting in high concentrations of DNA/vector complexes in endosomes in the cytosol. However, DNA/vector complexes have to be released from the endosomes to the cytoplasm. Furthermore, tracking the way of the complexes revealed that just a few complexes actually entered the nucleus (Branden et al., Nat. Biotechnol. 17 (1999), 784-787; Zelphati et al., Human Gene Ther. 10 (1999), 15-24). Thus, since successful gene therapy strongly depends on the efficient delivery of the polynucleotide to be introduced into the cytoplasm and the cell's nucleus, there is still a need to provide means which ensure a high transfection efficiency.

Thus, the technical problem underlying the present invention is to provide tools which allow for a highly efficient transfer of molecules, in particular nucleic acid molecules, into the cytoplasm and the nucleus of eukaryotic cells.

This problem is solved by the provision of the embodiments as characterized in the claims.

Thus, the present invention relates to a polypeptide comprising at least two peptide monomers, wherein each peptide monomer comprises an amino acid sequence which serves as a nuclear localization sequence or as a protein transduction domain in eukaryotic cells.

In this context the term “peptide” relates to a molecule containing at least two amino acid residues which are linked to each other by peptide bonds. Preferably, the amino acid residues are L-isomers. The amino acid residues may be naturally occurring amino acids or synthetic amino acids as well as modified amino acids and derivatives of naturally occurring amino acids. Such a peptide can be provided in different ways, e.g., by isolating it from naturally occurring sources, by expressing it from an appropriate recombinant nucleic acid molecule and purifying the resulting product by means and methods well known to the person skilled in the art or by chemical synthesis. The chemical synthesis is preferably but not exclusively carried out on solid phase according to standard procedures (“Boc”- or “Fmoc” chemistry (review: Atherton and Sheppard in: Solid phase peptide synthesis, IRL Oxford, University Press (1989)) using an automated peptide synthesizer. Derivatization steps or peptide cyclization may be carried out in fluid phase after cleavage of the peptide from the solid support.

The term “nuclear localization sequence” (NLS) means an amino acid sequence which induces transport of molecules comprising such sequences or linked to such sequences into the nucleus of eukaryotic cells. In this context the term “comprising” preferably means that the nuclear localization signal forms part of the molecule, i.e. that it is linked to the remaining parts of the molecule by covalent bonds. The term “linked” in this context means any possible linkage between the nuclear localization sequence and another molecule to be introduced into the nucleus of a eukaryotic cell, e.g., by covalent bonds, hydrogen bonds or ionic interactions.

The term “transport into the nucleus” in this context means that the molecule is translocated into the nucleus. Nuclear translocation can be detected by direct and indirect means: Direct observation by fluorescence or confocal laser scanning microscopy is possible when either or both the translocation inducing agent (the nuclear localization peptide) or the translocated molecule (e.g. the nucleic acid) are labeled with a fluorescent dye (labeling kits are commercially available, e.g. from Pierce or Molecular Probes). Translocation can also be assessed by electron microscopy if either or both the translocation inducing agent (the nuclear localization peptide) or the translocated molecule (e.g. the nucleic acid) are labeled with an electron-dense material such as colloidal gold (Oliver, Methods Mol. Biol. 115 (1999), 341-345). Translocation can be assessed in indirect ways if the transported molecule (e.g. nucleic acid) exerts a function in the nucleus. This function can be but is not limited to the expression of a gene encoded by the translocated nucleic acid including the consequences of such gene expression that may be exerted on other cellular molecules or processes. Such indirect actions include particularly the effect of direct transport of antisense oligonucleotides or ribozymes or the production of such molecules in the nucleus due to the translocation and expression of a nucleic acid sequence encoding such molecules. Preferably, the term “nuclear localization sequence” relates to an amino acid sequence which naturally occurs in a protein and which induces the transport of this protein into the nucleus of eucaryotic cells. Such amino acid sequences associate with cytoplasmic proteins (e.g. importin α und importin β) and the resulting complex binds to the nuclear pore, where a GTP consuming active transport translocates the complex through the nuclear pore into the nucleus. A multitude of nuclear localization sequences have been described. These include the nuclear localization sequence of the SV40 virus large T-antigen the minimal functional unit of which is the seven amino acid sequence PKKKRKV (SEQ ID NO: 1). Other examples of nuclear localization sequences include the nucleoplasmin bipartite NLS with the sequence NLSKRPAAIKKAGQAKKKK (SEQ ID NO: 2) (Michaud and Goldfarb, J. Cell Biol. 112 (1991), 215-223), the c-myct nuclear localization sequence having the amino acid sequence PAAKRVKLD (SEQ ID NO: 3) or RQRRNELKRSF (SEQ ID NO: 7) (Chesky et al., Mol. Cell Biol. 9 (1989), 2487-2492) and the hRNPA1 M9 nuclear localization sequence having the sequence NQSSNFGPMKGGNFGGRSSGPYGGGGQYFAKPRNQGGY (SEQ ID NO: 4) (Siomi und Dreyfuss, J. Cell Biol. 129 (1995), 551-560). Further examples for nuclear localization sequences are the sequence RMRKFKNKGKDTAELRRRRVEVSVELRKAKKDEQILKRRNV (SEQ ID NO: 8) of the IBB domain from importin-alpha (Gorlich et al., Nature 377 (1995), 246-248), the sequences VSRKRPRP (SEQ ID NO: 9) and PPKKARED (SEQ ID NO: 10) of the myoma T protein (Chelsky et al., loc. cit.), the sequence PQPKKKPL (SEQ ID NO: 11) of human p53 (Chelsky et al., loc. cit.), the sequence SALIKKKKKMAP (SEQ ID NO: 12) of mouse c-abl IV (Van Etten et al., Cell 58 (1989), 669-678), the sequences DRLRR (SEQ ID NO: 13) and PKQKKRK (SEQ ID NO: 14) of the influenza virus NS1 (Greenspan et al., J. Virol. 62 (1988), 3020-3026), the sequence RKLKKKIKKL (SEQ ID NO: 15) of the Hepatitis virus delta antigen (Chang et al., J. Virol. 66 (1992), 6019-6027) and the sequence REKKKFLKRR (SEQ ID NO: 16) of the mouse Mx1 protein (Zurcher et al., J. Virol. 66 (1992), 5059-5066). It is also possible to use bipartite nuclear localization sequences such as the sequence KRKGDEVDGVDEVAKKKSKK (SEQ ID NO: 17) of the human poly(ADP-ribose) polymerase (Schreiber et al., EMBO J. 11 (1992), 3263-3269) or the sequence RKCLQAGMNLEARKTKK (SEQ ID NO: 18) of the steroid hormone receptors (human) glucocorticoid (Cadepond et al., Exp. Cell Res. 201 (1992), 99-108).

The term “protein transduction domain” means an amino acid sequence which induces transport of proteins (i.e. β-galactosidase) comprising such sequence or linked to such sequence into the cytoplasm. In this context the term ,,comprising” preferably means that the protein transduction domain forms part of the molecule, i.e. that it is linked to the remaining parts of the molecule by covalent bonds. The term “linked” in this context means any possible linkage between the protein transduction domain sequence and another molecule to be introduced into the cytoplasm of a eucaryotic cell, e.g., by covalent bonds hydrogen bonds or ionic interactions.

The term “transport into the cytoplasm” in this context means that the molecule is translocated into the cytoplasm circumventing the endosomal pathway.

Preferably, the term “protein transduction domain” relates to an amino acid sequence which naturally occurs in a protein or is artificially designed and which induces the transport of this protein or itself into the cytoplasm of eucaryotic cells. Such amino acid sequences are receptor-independently delivered to the cytoplasm of eucaryotic cells. A multitude of protein transduction domains have been described which could be of basic or of hydrophobic character. These include the basic protein transduction domain of the HIV-1 TAT protein the minimal functional unit of which is the 11 amino acid sequence YGRKKRRQRRR (SEQ ID NO: 20). Other examples of basic protein transduction domains include the third helix of the Drosophila Antennapedia homebox gene with the sequence RQIKIWFQNRRMKWKK (SEQ ID NO: 21) (Derossi et al, J. Biol. Chem. 269 (1994), 10444-10450), the artificially designed protein transduction domains KRIHPRLTRSIR (SEQ ID NO: 22), PPRLRKRRQLNM (SEQ ID NO: 23), and RRQRRTSKLMKR (SEQ ID NO: 24); (Zhibao Mi et al., Molecular Therapy 2 (2000), 339-347). Examples for hydrophobic protein transduction domains include the sequence of transportan with the sequence GWTLNSAGYLLGKINLKALAALAKKIL (SEQ ID NO: 25) (Pooga M., The FASEB Journal 12 (1998), 67-77), AAVALLPAVLLALLAP (SEQ ID NO: 26), AAVLLPVLLAAP (SEQ ID NO: 27), and VTVLALGALAGVGVG (SEQ ID NO: 28) (Hawiger J., Current Opinion in Chemical Biology 3 (1999), 89-94).

Apart from a nuclear localization sequence or a protein transduction domain a monomer comprised in the polypeptide can of course contain further amino acid sequences, in particular sequences, which excert other functions.

The term “polypeptide” means a molecule consisting of peptides as defined above, except for homologous linear cationic polyaminoacids, such as poly-L-lysine, polyarginine and polyorinithine, which are preferably linked to each other by a peptide bond, or in the alternative via a disulfid bridge. Such a polypeptide—preferably has a length of at least 10, more preferably of at least 12 and even more preferably of at least 15 amino acid residues.

It has now been surprisingly found that the use of a polypeptide comprising at least two monomers comprising a nuclear localization sequence or a protein transduction domain drastically increases the efficiency of the transfer of attached molecules, in particular negatively charged molecules into the nucleus or cytoplasm of a eukaryotic cell. In this regard “attached” can mean e.g. covalently coupled or bound by electrostatic interaction. Although it has already been shown in the state of the art that nuclear localization sequences or protein transduction domains can be used to introduce DNA into the nucleus or cytoplasm of eukaryotic cells (see, e.g., WO 98/29541), it has unexpectedly been found that the direct repetition of such sequences in one polypeptide chain greatly enhances transfection efficiency, i.e. it results in an improved introduction of a molecule, in particular of a nucleic acid molecule into the nucleus and cytoplasm of eukaryotic cells. The term “improved introduction” in this context means a more efficient uptake of a molecule by cells in the presence of a multimerized nuclear localization sequence or of a protein transduction domain when compared to the situation where only a monomer of such a nuclear localization sequence or protein transduction domain is used or multimers, which are however not located in the same polypeptide. This can be determined by comparing the amount of the molecule translocated into the nucleus under the different conditions, preferably, in the case of nucleic acid molecules, by determining the expression of the introduced nucleic acid molecule in the cells.

The term “molecule” in this context can mean any kind of molecule to be introduced into the nucleus in order to excert a function. Function in this regard means in particular modulation of the expression of a gene, wherein the gene can be an endogenous gene or a foreign gene introduced into the nucleus (exogenous gene). Modulation can be, e.g., inhibition or induction of expression. Function can also mean influencing the cell division process or chromatin structure and function. The term “negatively charged molecule” refers to any kind of negatively charged molecule which may be introduced into a cell, preferably to polypeptides, hormones, e.g. peptide hormones, steroid hormones, or thyroid hormones. The molecule can in particular be a molecule which is an inhibitor or activator of an enzymatic activity in the nucleus. In a preferred embodiment the negatively charged molecule is a nucleic acid molecule.

In a preferred embodiment of the polypeptide according to the invention the nuclear localization sequence comprises an amino acid sequence selected from the group consisting of

(SEQ ID NO:1)

| | |
|---|---|
| (a) | PKKKRKV; |

(SEQ ID NO:5)

| | |
|---|---|
| (b) | PKKKRKVG; |

(SEQ ID NO:2)

| | |
|---|---|
| (c) | NLSKRPAAIKKAGQAKKKK; |

(SEQ ID NO:4)

| | |
|---|---|
| (d) | NQSSNFGPMKGGNFGGRSSGPYGGGGQYFAKPRNQGGY; |
| | and |

(SEQ ID NO:3)

| | |
|---|---|
| (e) | PAAKRVKLD. |

The nuclear localization sequence present in the monomers of the polypeptide of the invention may be identical to each other, but they can also differ from each other. I.e. it is possible to have a polypeptide in which every monomer comprises the same nuclear localization sequence. But it is also possible to have polypeptides in which the different monomers comprise different nuclear localization sequences. In this regard all conceivable combinations are possible, namely polypeptides having one or more monomers with one nuclear localization sequence and one or more monomers with one or more other nuclear localization sequences.

In a particularly preferred embodiment the polypeptide is the tetramer (PKKKRKV)₄ or (PKKKRKVG)₄.

In a preferred embodiment of the polypeptide according to the invention the protein transduction domain comprises an amino acid sequence selected from the group consisting of

| | | |
|---|---|---|
| (a) | YGRKKRRQRRR; | (SEQ ID NO:20) |
| (b) | KRIHPRLTRSIR; | (SEQ ID NO:22) |
| (c) | PPRLRKRRQLNM; | (SEQ ID NO:23) |
| (d) | RRQRRTSKLMKR; | (SEQ ID NO:24) |
| (e) | RQIKIWFQNRRMKWKK | (SEQ ID NO:21) |
| (f) | KLALKLALKALKAALKLA | (SEQ ID NO:29) |
| (g) | GWTLNSAGYLLGKINLKALAALAKKIL | (SEQ ID NO:25) |
| (h) | AAVALLPAVLLALLAP | (SEQ ID NO:26) |
| (i) | AAVLLPVLLAAP, | (SEQ ID NO:27) |
| | and | |
| (j) | VTVLALGALAGVGVG. | (SEQ ID NO:28) |

The protein transduction domain present in the monomers of the polypeptide of the invention may be identical to each other, but they can also differ from each other. I.e. it is possible to have a polypeptide in which every monomer comprises the same protein transduction domain. But it is also possible to have polypeptides in which the different monomers comprise different nuclear localization sequences. In this regard all conceivable combinations are possible, namely polypeptides having one or more monomers with one protein transduction domain and one or more monomers with one or more other protein transduction domains.

In a particularly preferred embodiment the polypeptide is the dimer, trimer and tetramer C(YGRKKRRQRRRG)₂₋₄ (SEQ ID NO: 30, 31 and 32, respectively).

The peptide of the present invention may also comprise a combination of at least two peptide monomers wherein at least one peptide monomer comprises a nuclear localization sequence and wherein at least one monomer comprises a protein transduction domain.

In general, the polypeptide of the invention comprises at least two monomers. Preferably, it comprises at least three monomers, more preferably at least four monomers, even more preferably at least five monomers and particularly preferred at least ten monomers. In general there is no upper limit for the number of monomers comprised in the polypeptide according to the invention. However, it is preferred that the polypeptide does not comprise more than 30 monomers, more preferably not more than 25 monomers, even more preferably not more than 20 monomers and particularly preferred not more than 15 monomers.

The present invention also relates to polypeptide conjugates which comprise at least two polypeptides according to the invention which are covalently linked to each other, preferably but not exclusively by amide, disulfide, ester, ether, thioether, sulfonamide, other thiol bonds such as thioureas, hydrazides and Schiffs base bonds, more generally carbon-nitrogen single bonds and carbon-nitrogen double bonds. Such bonds can be introduced in a variety of ways and are well known to the person skilled in the art of chemical synthesis. Such methods are reviewed in text books and various review papers (e.g. Brinkley, Bioconj. Chem. 3 (1992), 2-13; Wong and Wong, Enzyme Microb. Technol. 14 (1992), 866-874). Furthermore, commercially available bifunctional crosslinkers may be used (e.g. from Pierce).

In a preferred embodiment the polypeptides or polypeptide conjugates according to the invention are further modified insofar as they are linked, covalently or non-covalently, to another molecule which exerts an effector function on or in the target cell. Such a molecule can be a receptor ligand or an antibody which allows attachment to the target cell surface. Receptor ligands may be chosen from natural sources such as transferrin or various asialoglycoproteins or of synthetic origins such as synthetic peptides fitting binding sites of known receptors (as for example described by Erbacher et al., Gene Ther. 6 (1999), 138-145; Plank et al., Bioconj. Chem. 3 (1992), 533-539; Wu and Wu, J. Biol. Chem. 262 (1987), 4429-4432). The use of receptor ligands or antibodies is not limited to particlur types of ligands or antibodies and is solely determined by the presence of a binding partner on the envisaged target cell population. Furthermore the effector molecule may be drug supposed to exert it's function in the nucleus. Such drugs include for example specific antibodies to nuclear factors involved in the transcription of particular genes.

Methods for linking such molecules to the polypeptide or polypeptide conjugate of the present invention are well known in the art and include the use of bifunctional crosslinkers such as described by Brinkley (loc. cit.) and Wong and Wong, (loc. cit.) which may be of commercial origin (e.g. Pierce).

The present invention also relates to complexes comprising at least one polypeptide and/or at least one polypeptide conjugate according to the invention and at least one molecule to be introduced into the cells, preferably a nucleic acid molecule. Preferably, the polypeptide and/or polypeptide conjugate and the molecule, e.g. the nucleic acid molecule, in such a complex interact by ionic bonds. The preparation of such complexes is well known in the art and is described, e.g., in Plank et al., (J. Biol. Chem. 269 (1994), 12918-12924) and Trubetskoy et al. (Nucl. Acids Res. 27 (1999), 3090-3095).

In a preferred embodiment the complex according to the invention is, preferably covalently or by ionic bonding, linked to another molecule which allows cytoplasmic delivery as a first step before nuclear translocation. Such molecules can, e.g., be membrane-destabilizing peptides such as those derived from influenza virus hemaglutinin and those derived from other sources such as reviewed by Plank et al. (Advanced Drug Delivery Reviews 34 (1998), 21-35).

Another example for a molecule which can be linked to the complex by covalent or electrostatic interaction is polyethylene glycol in order to exert a protective and stabilizing effect on the complex during the delivery phase in vivo and in vitro (Ogris et al., Gene Therapy 6 (1999), 595-605; Finsinger et al., Gene Therapy 7 (2000), 1183-1192).

Furthermore, the present invention relates to a process for preparing a complex according to the invention comprising the step of contacting the polypeptide and/or polypeptide conjugate according to the invention with a molecule, e.g. a nucleic acid molecule, under conditions which allow the formation of the complex. The person skilled in the art will recognize that the specific conditions necessary for the formation of the complex depends on the specific nature of the polypeptide and/or polypeptide conjugate and the molecule. However, adjusting the conditions lies well within the skill of the person skilled in the art (Ogris et al., Gene Ther. 5 (1998), 1425-1433; Trubetskoy et al., Anal. Biochem. 267 (1999), 309-313) for example. The molecule present in the complex can be a molecule as described above.

The nucleic acid molecule present in the complex according to the invention can be any possible nucleic acid molecule, i.e. DNA or RNA, or DNA/RNA hybrids, single stranded or double stranded DNA, oligonucleotides, linear or circular, natural or synthetic, modified or not. Preferably, the nucleic acid molecule comprises a region encoding a gene product, e.g., a transcribable or a not-transcribable RNA. Particularly preferred the nucleic acid molecule encodes a polypeptide or an antisense oligonucleotide sequence or a ribozyme. Furthermore the nucleic acid molecule can be an antisense oligonucleotide or a ribozyme itself.

The polypeptide and/or peptide conjugate and/or complex according to the invention can furthermore also be combined with particulate drug delivery systems for introducing them into cells such as, e.g. magnetic particles, silica beads, PLGA, nano- or microspheres, chitosan etc.

The present invention also relates to a pharmaceutical composition comprising a polypeptide and/or polypeptide conjugate and/or complex according to the present invention.

The pharmaceutical composition of the present invention may optionally comprise a pharmaceutically acceptable carrier, excipient and/or diluent. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Proteinaceous pharmaceutically active matter may be present in amounts between 1 ng and 10 mg per dose; however, doses below or above this exemplary range are envisioned, especially considering the aforementioned factors. Administration of the suitable compositions may be effected by different ways, e.g., by intravenous, intraperitoneal, subcutaneous, intramuscular, topical or intradermal administration. If the regimen is a continuous infusion, it should also be in the range of 1 μg to 10 mg units per kilogram of body weight per minute, respectively. Progress can be monitored by periodic assessment. The compositions of the invention may be administered locally or systemically. Administration will generally be parenterally, e.g., intravenously. The compositions of the invention may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins, interferons and/or CpG-containing DNA stretches depending on the intended use of the pharmaceutical composition.

Furthermore, the present invention relates to a process transferring a molecule, e.g. a nucleic acid molecule, into the nucleus of a eukaryotic cell comprising the step of contacting the cell with

(i) a polypeptide and/or polypeptide conjugate according to the invention in the presence of the molecule; and/or

(ii) the complex according to the invention; and/or

(iii) the pharmaceutical composition according to the invention.

This process may be applied by direct administration of the polypeptide, polypeptide conjugate, complex and/or pharmaceutical composition to cells of a eukaryotic organism in vivo, or by in vitro treatment of cells, e.g., by the treatment of cells which can be extracted from the organism and are then re-introduced into the organism (ex vivo process).

In a preferred embodiment the process according to the invention is for transferring a nucleic molecule into a vertebrate tissue. These tissues include those of muscle, skin, brain, lung, liver, spleen, bone marrow, thymus, heart, lymph, bone, cartilage, pancreas, kidney, gall bladder, stomach, intestine, testis, ovary, uterus, rectum, nervous system, eye, gland, connective tissue, blood, tumor etc. In case of an in vivo application the administration of the polypeptide, polypeptide conjugate, complex and/or pharmaceutical composition may be made, e.g., by intradermal, subdermal, intravenous, intramuscular, intranasal, intracerebral, intratracheal, intraarterial, intraperitoneal, intrapleural, intracoronary or intratumoral injection, with a syringe or other devices such as catheters. Furthermore transdermal administration, inhalation or aerosol administration are contemplated as well as electroporation. Electroporation may be exploited for cytoplasmic delivery prior to nuclear translocation and may be used to assist all of the above-mentioned routes of administration.

The present invention furthermore relates to a kit comprising the polypeptide, polypeptide conjugate or complex according to the invention. Such a kit may furthermore comprise a molecule, e.g. a nucleic acid molecule to be introduced into cells, a buffer allowing for complexation between the polypeptide or polypeptide conjugate and a molecule, e.g. a nucleic acid molecule, and/or instructions for carrying out the method according to the invention for transferring a molecule, e.g. a nucleic acid molecule into a eukaryotic cell.

Advantageously, the kit of the present invention further comprises, optionally (a) reaction buffer(s), storage solutions and/or remaining reagents or materials required for the conduct of scientific assays or the like. Furthermore, parts of the kit of the invention can be packaged individually in vials or bottles or in combination in containers or multicontainer units.

Moreover, the present invention relates to the use of a polypeptide, polypeptide conjugate, complex and/or pharmaceutical composition according to the invention for transferring a molecule, e.g. a nucleic acid molecule into eukaryotic cells, in particular into the nucleus.

The invention has been described in an illustrative manner, and it is to be understood that the terminology which has been used is intended to be in the nature of words of description rather than of limitation. Obviously, any modifications and variations of the present invention are possible in light of the above teachings. It is therefore to be understood that within the scope of the claims, the invention may be practiced otherwise than as specifically described.

All of the above cited disclosures of patents, publications and data base entries are specifically incorporated herein by reference in their entirety to the same extent as if each such individual patent, publication or entry were specifically and individually indicated to be incorporated by reference.

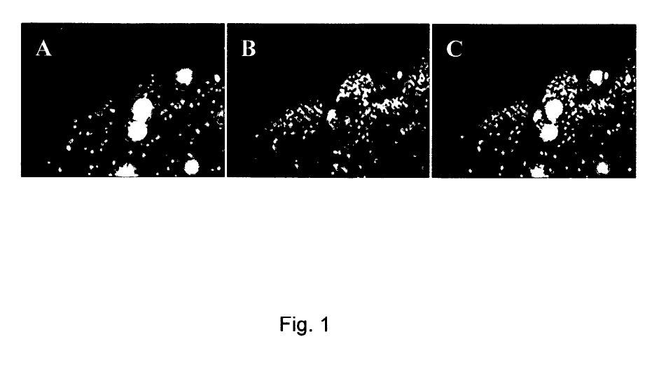

FIG. 1 shows the nuclear transport of BSA covalently linked to (PKKKRKVG)₄. HeLA S6 cells were permeabilized for 2 min with 40 μM digitonin in transport buffer followed by incubation with 600 nM (PKKKRKVG)₄/BSA-BODIPY and equivalent amounts of BSA-Texas Red for 30 min. Cells were fixed with 4% formaldehyde and evaluated under fluorescence microscope. A-C represent the same microscopic field. A) Green fluorescence of BSA-BODIPY. B) Red fluorescence of BSA-Texas Red. C) Resulting image with both fluorescence dyes.

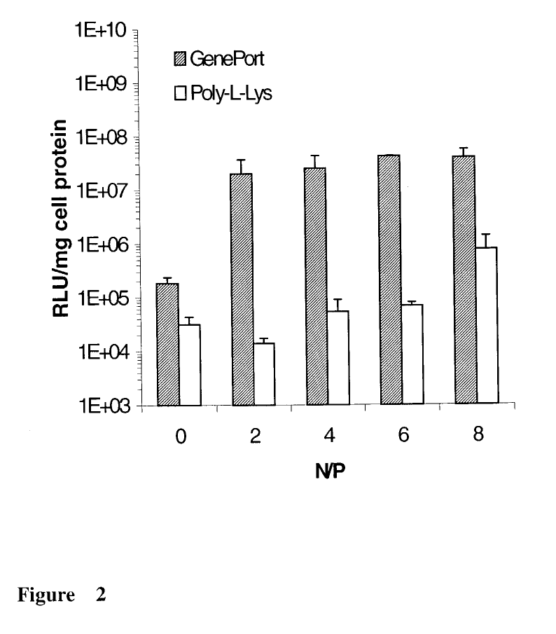

FIG. 2 shows transfection of 16HBE14o-cells with poly-L-lysine and (PKKKRKVG)₄. Cells were transfected with 1 μg CMVL-W complexed with increasing amounts of (PKKKRKVG)₄ or poly-L-lysine 2.9 kD. Luciferase activity was measured (10 sec) after 24 h.

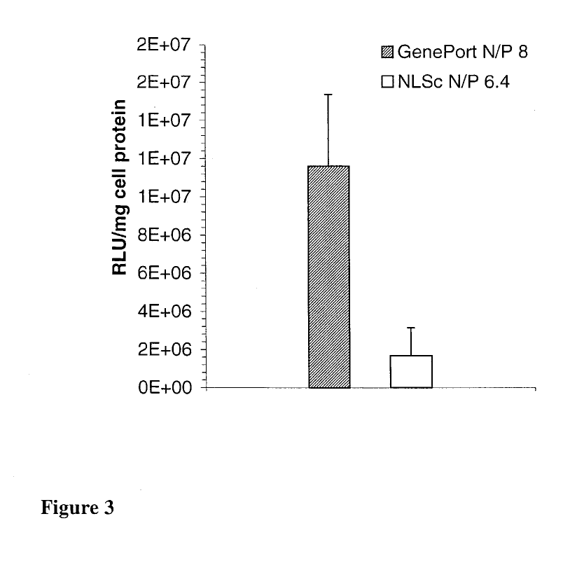

FIG. 3 shows transfection of 16HBE14o-cells with (PKKKRKVG)₄ and mNLS. Cells were transfected with 1 μg CMVL-W complexed with 4.8 μg (PKKKRKVG)₄ (NIP 8) or 4.8 μg mNLS (NIP 6.4). Luciferase activity was measured (10 sec) after 24 h.

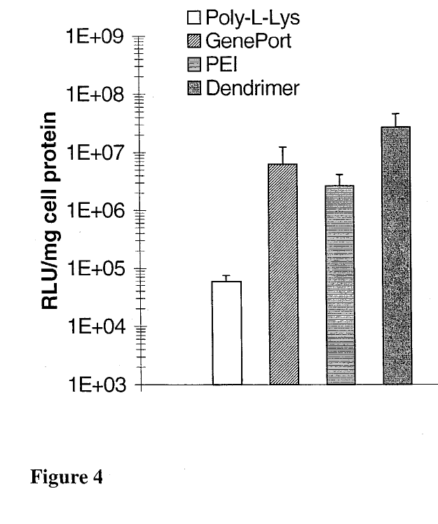

FIG. 4 shows transfection of 16HBE14o-cells with different non-viral vectors. Cells were transfected with 1 μg CMVL-W complexed with 5 μg poly-L-lysine 2.9 kDa (N/P 8), 4.8 μg (PKKKRKVG)₄ (N/P 8), 1.96 μg PEI (N/P 5) or 3.96 μg fractured Dendrimer (N/P 4.5). Luciferase activity was measured (10 sec) after 24 h.

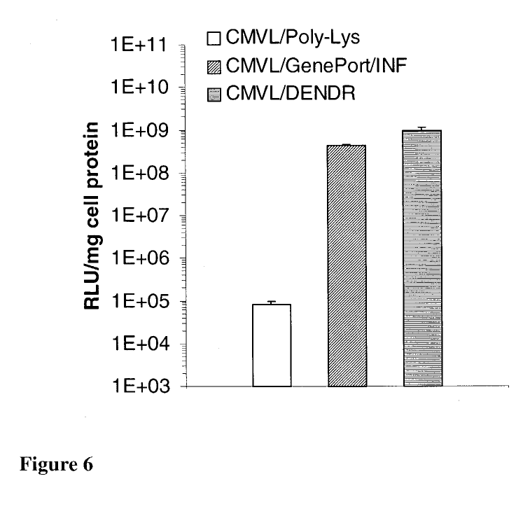

FIG. 6 shows the use of endosomolytic agents for transfection of 16HBE14o-cells with 1 μg CMVL-W complexed with 4.8 μg (PKKKRKVG)₄ (N/P 8) or with additional 0.78 μg influenca peptide (INF7a). Luciferase activity was measured (10 sec) after 24 h.

FIG. 6 shows the comparison of transfection efficiency of different non-viral vectors. Cells were transfected with 1 μg CMVL-W complexed with 5 μg poly-L-lysine 2.9 kD (N/P 8), 4.8 μg (PKKKRKVG)₄ (N/P 8) with 0.78 μg INF7a and 3.96 μg fractured Dendrimer. Luciferase activity was measured (10 sec) after 48 h.

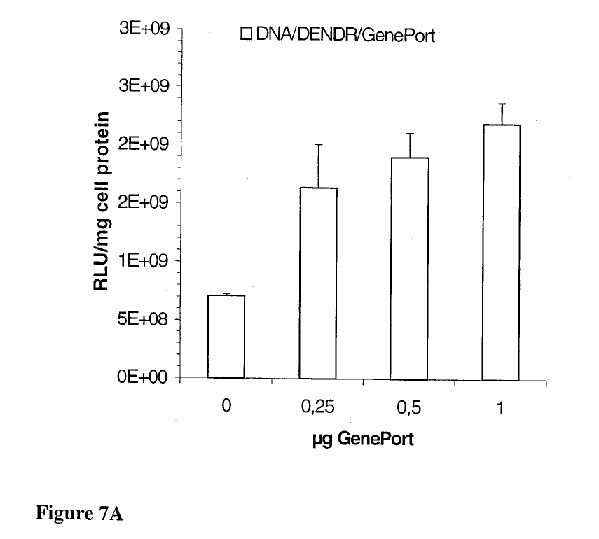

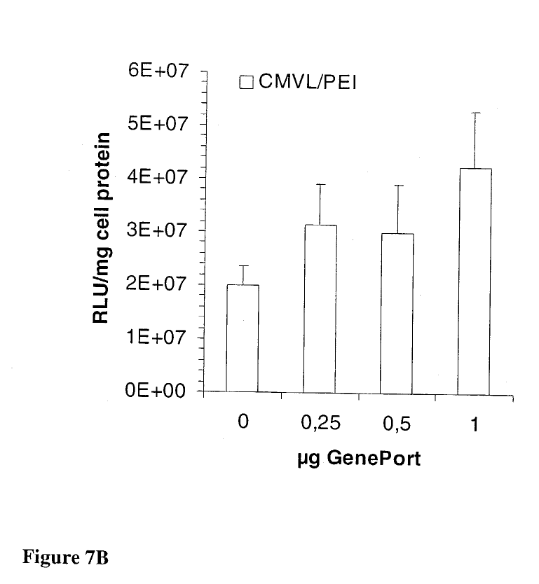

FIG. 7 shows the enhancement of polyfection with (PKKKRKVG)₄. 16HBE14o-cells were transfected with 1 μg CMVL-W complexed with 3.96 μg fractured Dendrimer (N/P 4.5) or 1.96 μg PEI (N/P 5) and additional with (PKKKRKVG)₄ in increasing concentration. Luciferase activity was measured (10 sec) after 24 h.

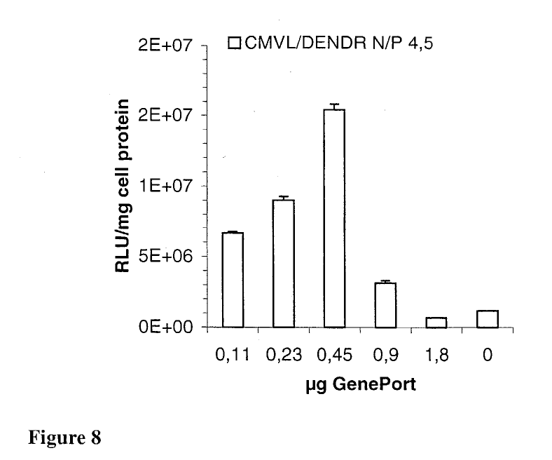

FIG. 8 shows transfection of 100% confluent cells with CMVL-W/Dendrimer/(PKKKRKVG)₄ complexes. 100% confluent 16HBE14o-cells were transfected with 1 μg CMVL-W complexed with 3.96 μg fractured Dendrimer and (PKKKRKVG)₄ in increasing concentration. Luciferase activity was measured (10 sec) after 24 h.

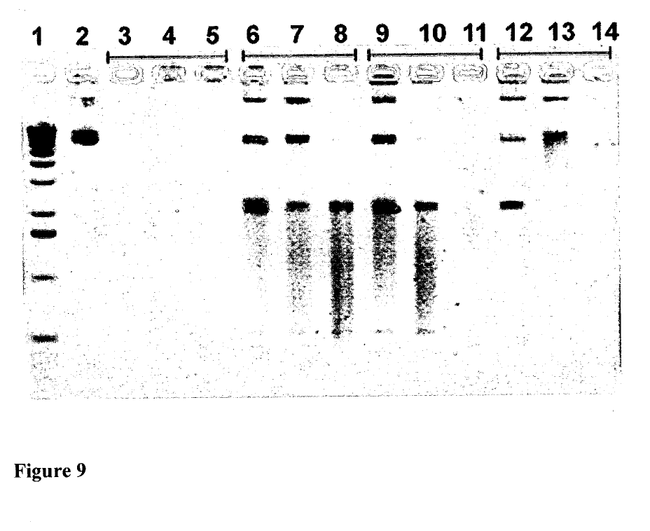

FIG. 9 shows the comparison of the stability of DNA/(PKKKRKVG)₄-complexes to DNA/Dendrimer complexes. Digestion of the complexes was carried out for 1 hour at 37° C. with increasing activity of DNase I.

| 1 = 1 kb standard (600 ng) (GIBCO #15615) | |
|---|---|
| 2 = DNA (pEGFP) without DNase I (450 ng) | |
| 3 = DNA (pEGFP) | +2.5 U DNase I (450 ng) |
| 4 = | +5 U DNase I |
| 5 = | +10 U DNase I |
| 6 = DNA (3 μg)/GenePort N/P = 8 | +2.5 U DNase I |
| 7 = | +5 U DNase I |
| 8 = | +10 U DNase I |
| 9 = DNA (3 μg)/Dendrimer N/P = 4.5 | +2.5 U DNase I |
| 10 = | +5 U DNase I |
| 11 = | +10 U DNase I |
| 12 = DNA (3 μg)/Dendrimer N/P = 4.5 + | |
| 1 μg Gene Port | +2.5 U DNase I |
| 13 = | +5 U DNase I |
| 14 = | +10 U DNase I |

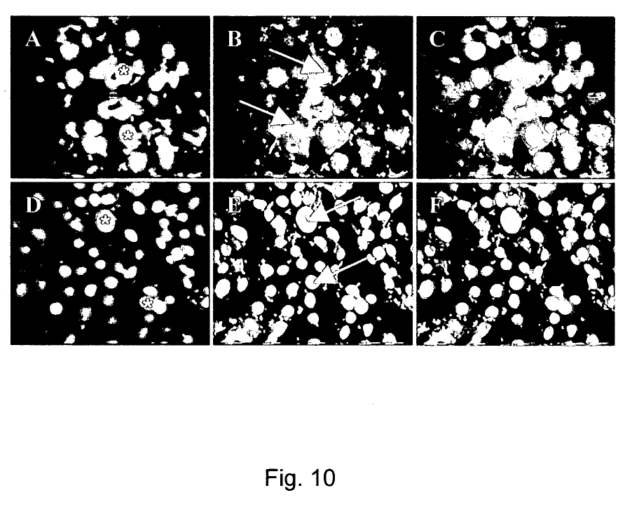

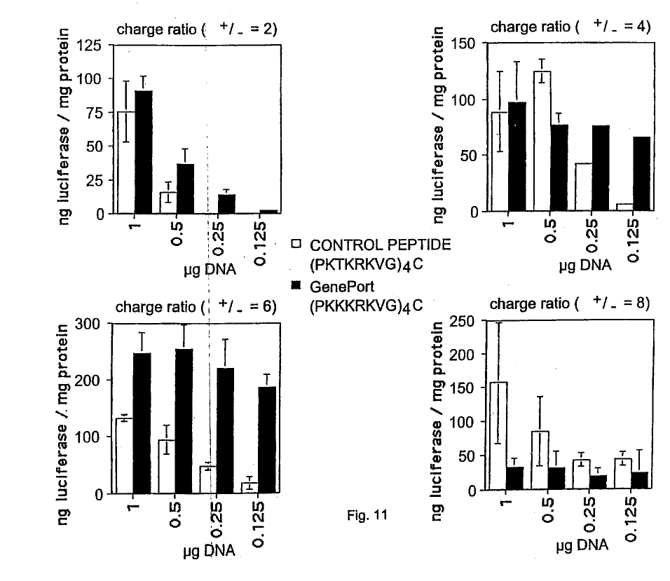

FIG. 10 shows the tracking of the way of DNA-TOTO-3/(PKKKRKVG)₄-FITC complexes on their way to the nucleus. 16HBE14o-cells were transfected with 5 μg CMVL-W and 24 μg (PKKKRKVG)₄ (N/P 8). Transfections were stopped after 4 h (A-C) and 30 h (D-F) by fixation with 4% formaldehyde. A+D: Green fluorescence of (PKKKRKVG)₄-FITC. B+E: Red fluorescence of DNA-TOTO-3. C+F: resulting images.

FIG. 11 (PKKKRKVG)₄C enhances gene delivery when used to form a DNA complex compared to complexes prepared with the control peptide (PKTKRKVG)₄C depending on complex formulation. At higher charge ratios (e.g. ⁺/₋=8) the control complex is superior in the concentration range examined. This can be explained by competition of free NLS peptide (not part of the DNA complex) for binding to the nuclear translocation machinery which the control peptide is not able to do. In fact example 8 demonstrates that only a limited amount of (PKKKRKVG)₄C can be associated with DNA.

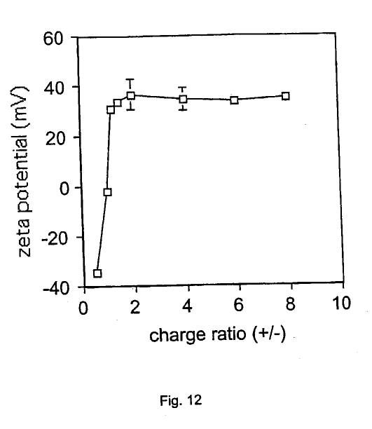

FIG. 12 Under the experimental conditions DNA can associate the cationic peptide only up to a charge ratio of 2. Above this charge ratio the zeta potential which increases with increasing amounts of peptide up to this point reaches a plateau. The zeta potential is a measure of the surface charge of particles.

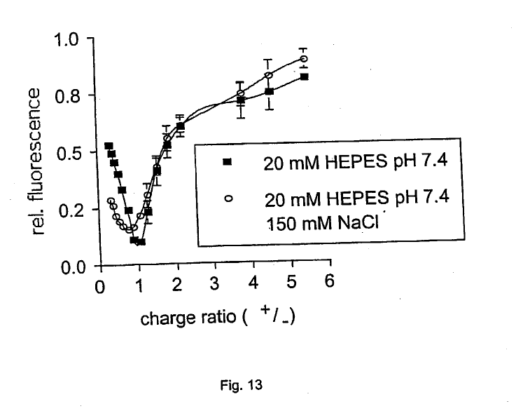

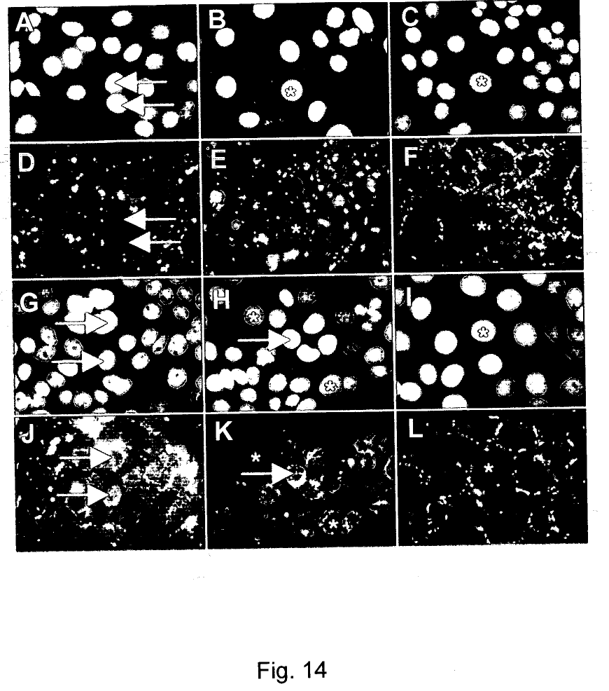

FIG. 13 At charge ratios below 1 fluorescence decreases or quenching increases due to DNA compaction resulting in particle formation. Minimum fluorescence is observed at the point of optimal achievable compaction. Beyond this point compaction may still be complete, however not all fluorescent peptide is in the interior of the vector particle. Hence, fluorescence increases again.

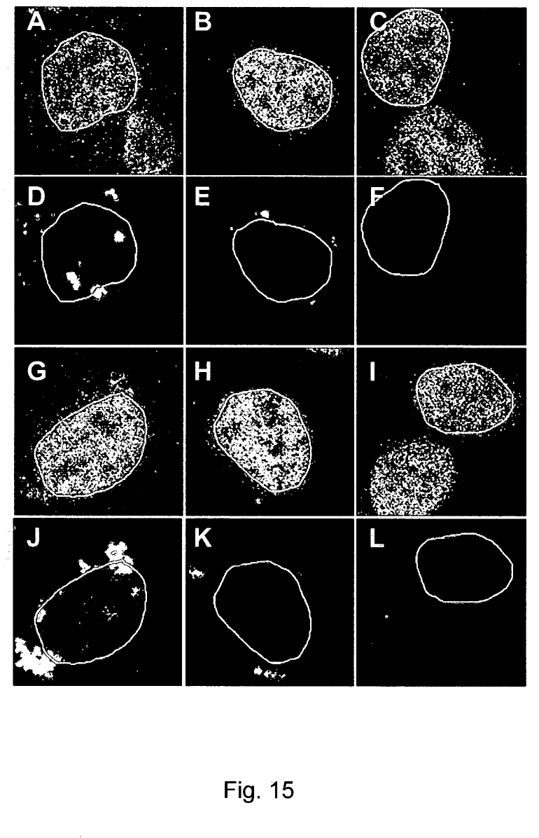

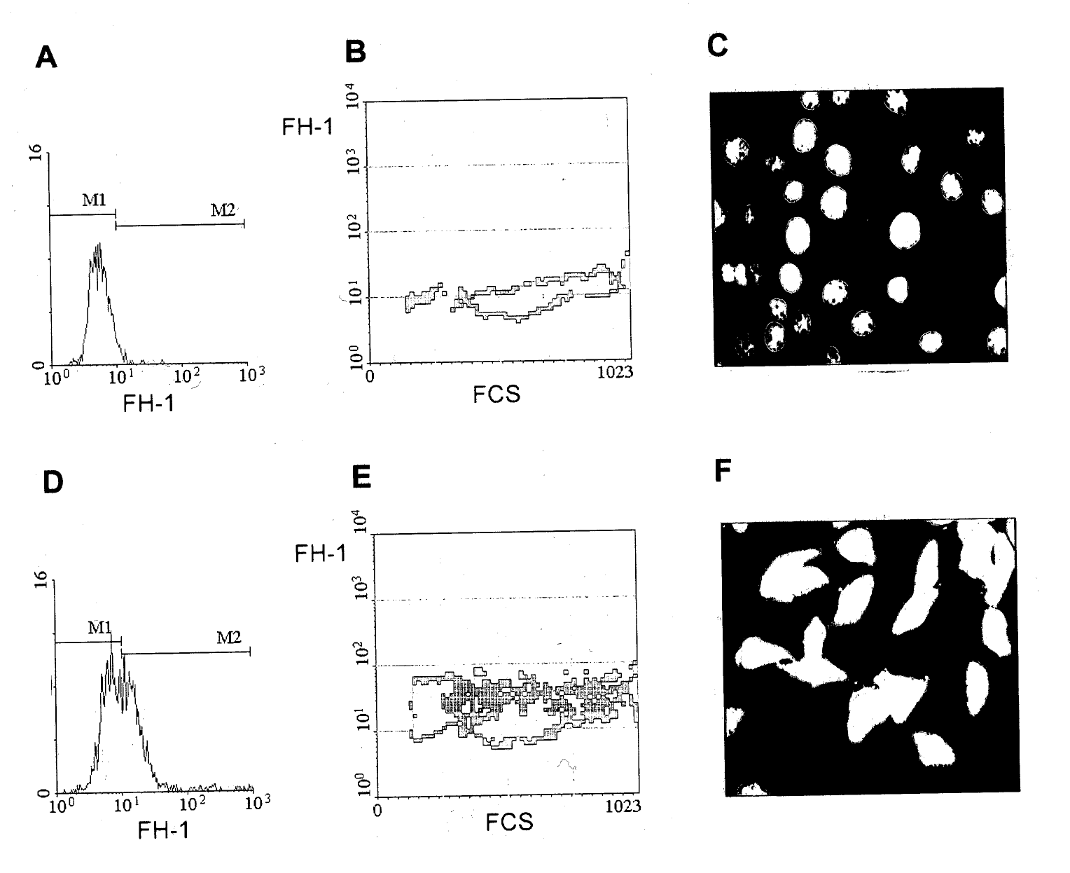

FIG. 14 shows the distribution of plasmid DNA after cell transfection. HeLa S6 cells were transfected with pEGFP. Transfection was stopped at 2 hours (A-F) and 24 hours (G-L). (D,J) Cells were transfected with pEGFP/(PKKKRKVG)₄ complexes. (E,K) Transfection with pEGFP/(PKTKRKVG)₄ complexes. (F,L) Transfection with naked DNA (pEGFP). Plasmid DNA was localized by FISH. The images were generated with a 40× objective by fluorescence microscopy. Blue signals represent the cell nuclei stained with DAPI, red signals show the distribution of pEGFP in the same microscope field, using a digoxigenin labeled DNA probe. The probe was detected with anti digoxigenin rhodamine antibody. See FIG. 15 for corresponding images by confocal laser scanning microscopy. * no plasmid DNA in the nucleus.→cellular distribution of plasmid DNA.

FIG. 15 shows the intracellular localization of plasmid DNA. Using the same microscope slides as in FIG. 14 images were generated with a 63× objective by confocal laser scanning microscopy. (D,J) Cells were transfected with pEGFP/(PKKKRKVG)₄ complexes. (E,K) Transfection with pEGFP/(PKTKRKVG)₄ complexes. (F,L) Transfection with naked DNA (pEGFP). The green signal represents the cell nuclei stained with Sytox 16, the red signal shows the distribution of pEGFP in the same microscope field using a digoxigenin labeled DNA probe. The probe was detected with anti digoxiaenin rhodamine antibody.—nuclear outline.

FIG. 16 shows the proportion of transgene expressing cells after transfection with (PKKKRKVG)₄. (A, B, D, E) Flow cytometry of 16HBE14o-cells transfected with DNA (pEGFP) or DNA complexed with (PKKKRKVG)₄. Transfection was stopped after 24 hours. Cells were treated with trypsin and resuspended in medium. FCS: Forward scatter. FH-1: green channel for the GFP signal. (A, B) Control. Cells were only transfected with 1 μg pEGFP. Mean: 5.66. M1: 96% of total cells. M2: 4% of total cells. (D, E) Transfection with 1 μg pEGFP/(PKKKRKVG)₄ complexes (N/P 8). Mean: 10.25. M1: 55% of total cells. M2: 45% of total cells. Near half the cell population expresses green fluorescence protein. (C, F) Fluorescence microscopy images of HeLa S6 cells transfected with (C) 1 μg pEGFP or (F) 1 μg pEGFP complexed with (PKKKRKVG)₄ (N/P 8). Images were taken with a 40× objective (exposure time: 2 sec).

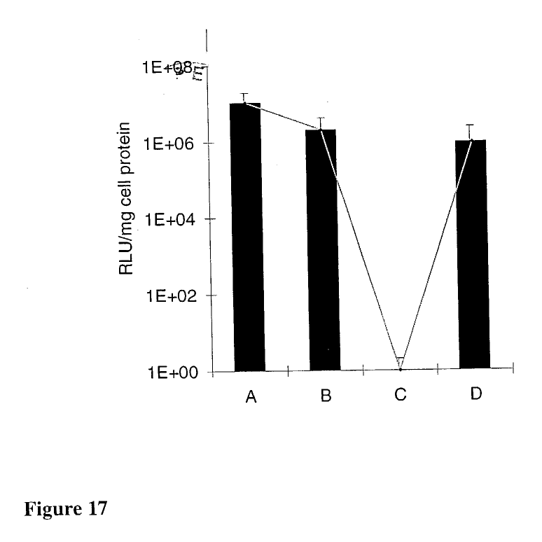

FIG. 17 shows the inhibition of gene transfer. 16HBE14o-cells were transfected with 1 μg CMVL/(PKKKRKVG)₄ complexes (N/P8). A 30-fold molar excess of (PKKKRKVG)₄ was added to the cells prior to complex addition. (A) no free (PKKKRKVG)₄. (B) free (PKKKRKVG)₄ and DNA/(PKKKRKVG)₄ complexes were added at the same time. (C) free (PKKKRKVG)₄ was added to the cells 20 min before the DNA/(PKKKRKVG)₄ complexes were added. (D) free (PKKKRKVG)₄ was added to the cells 45 min before the DNA/(PKKKRKVG)₄ complexes were added. Luciferase activity was measured (10 sec) after 24 h.

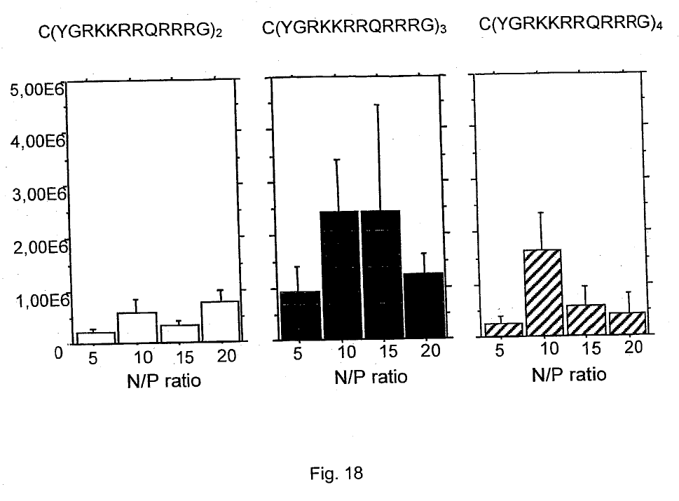

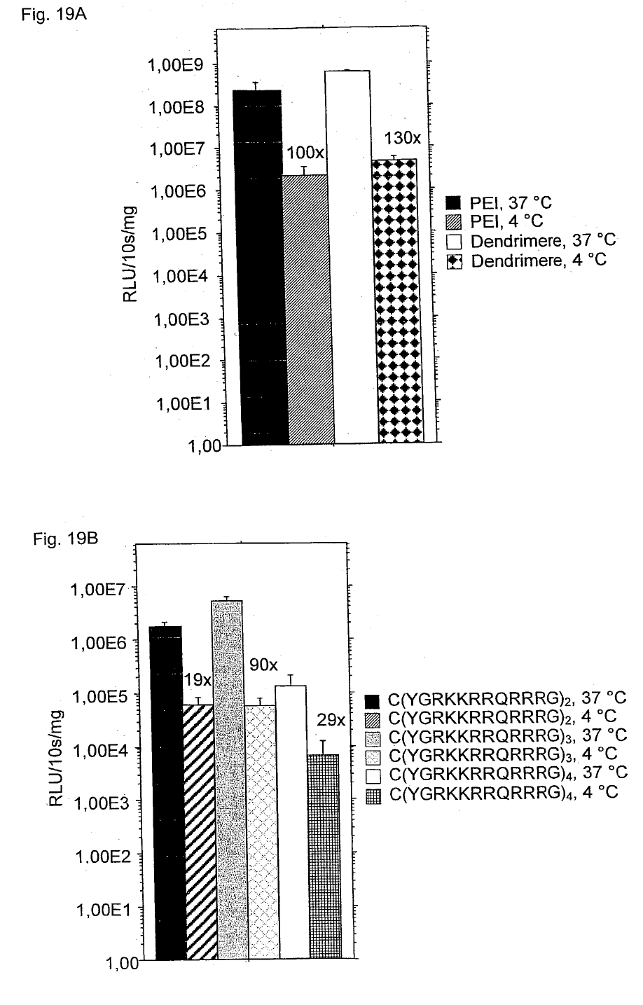

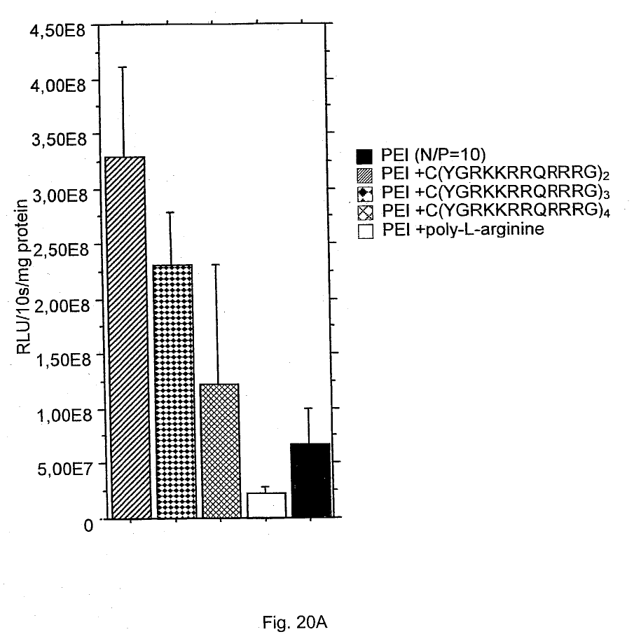

FIG. 18 shows the transfection of COS7 cells with C(YGRKKRRQRRRG)₂₄ Cells were transfected with 1 μg of CMVL-W complexed with increasing amounts of C(YGRKKRRQRPRG)₂. Luciferase activity was measured (10 sec) after 24 h.

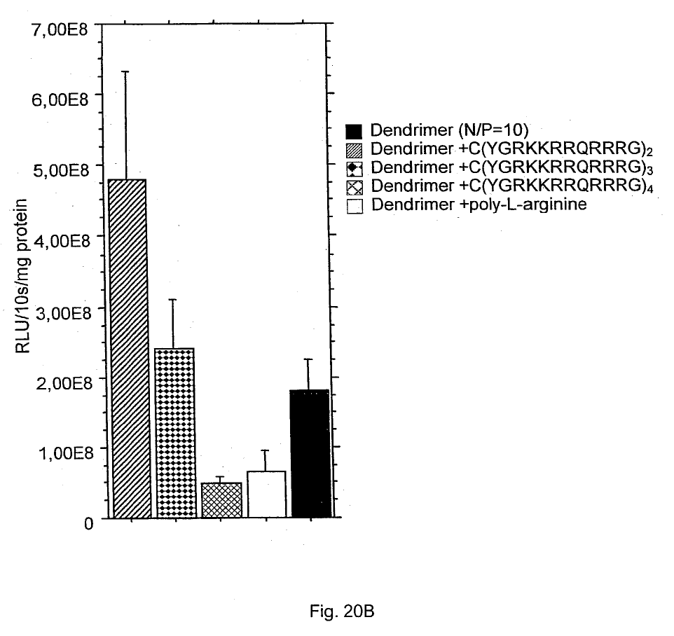

FIG. 19 shows the transfection of COS7 cells with C(YGRKKRRQRRRG)₂₄ at 4° C. in comparison with 37° C. Cells were transfected with 1 μg of CMVL-W complexed with C(YGRKKRRQRRRG)₂₄ at N/P=10 for 4 h at 4° C. or at 37° C. Luciferase activity was measured (10 sec) after 24 h.

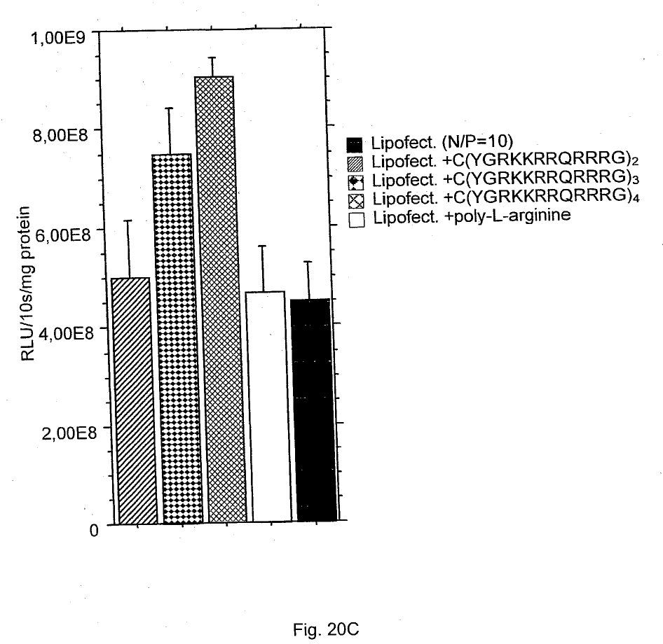

FIG. 20a shows the effect of C(YGRKKRRQRRRG)₂₄ and poly-L-arginine on polyethylenimine 25 kDa mediated gene transfer. 1 μg of CMVL-W Plasmid DNA was first complexed with C(YGRKKRRQRRRG)₂₄ or poly-L-arginine at N/P=1 respectively and subsequently polyethylenimine 25 kDa was added (N/P=10). The resulting complexes were used for transfection on COS7 cells. Luciferase activity was measured (10 sec) after 24 h.

FIG. 20b shows the effect of C(YGRKKRRQRRRG)₂₄ and poly-L-arginine on fractured Dendrimers mediated gene transfer. 1 μg of CMVL-W Plasmid DNA was first complexed with C(YGRKKRRQRRRG)₂₄ or poly-L-arginine at N/P=1 respectively and subsequently fractured Dendrimers were added (N/P=4,5). The resulting complexes were used for transfection on COS7 cells. Luciferase activity was measured (10 sec) after 24 h.

FIG. 20c shows the effect of C(YGRKKRRQRRRG)₂4 and poly-L-arginine on Lipofectamine mediated gene transfer. 1 μg of CMVL-W Plasmid DNA was first complexed with C(YGRKKRRQRRRG)₂₄ or poly-L-arginine at N/P=1 respectively and subsequently Lipofectamine was added (w/w 1/10)). The resulting complexes were used for transfection on COS7 cells. Luciferase activity was measured (10 sec) after 24 h.

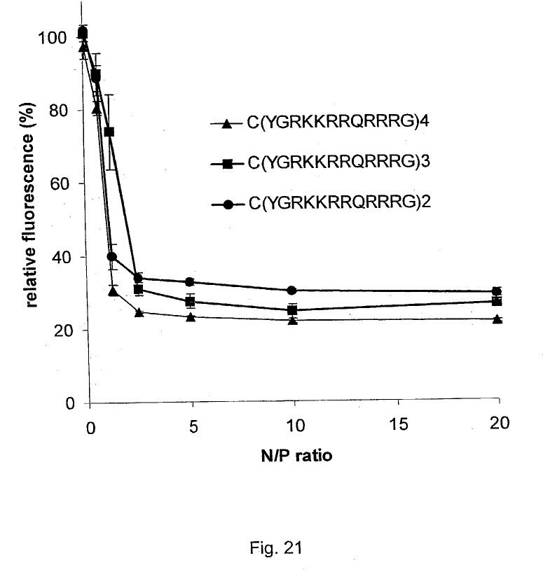

FIG. 21 shows the ability of C(YGRKKRRQRRRG)₂₋₄ to condense DNA. DNA was labeled with TOTO-1 (every 20 base pairs) and complexes were prepared with increasing amounts of C(YGRKKRRQRRRG)₂₋₄ at indicated N/P ratios. Fluorescence was measured and compared to fluorescence emitted when labeled DNA was not complexed.

The following examples illustrate the invention.

### EXAMPLE 1

### Synthesis and Testing of a Gene Transfer Enhancing Signal

Small polypeptides for gene delivery were designated for in vivo studies. For this purpose, a seven amino acid long sequence of the NLS of the large T-antigen of SV40, having the amino acid sequence PKKKRKV (SEQ ID NO: 1), was chosen. To achieve enough positive charges for a stable electrostatic complexation of DNA the NLS sequence was prolonged by repeating the seven amino acid sequence to a 4.4 kD protein. To gain more flexibility in the three dimensional structure of the peptide, a glycin was added at the end of each NLS. The following peptides were synthesized on an Applied Biosystems 431 A automatic synthesizer: (PKKKRKVG)₄ (SEQ ID NO: 5) containing the wild-type large T-antigen NLS and (PKTKRKVG)₄ (SEQ ID NO: 6) (mNLS) containing mutant large T-NLS (the threonin mutant is known to be transport deficient).

In order to determine whether the polypeptide (PKKKRKVG)₄ is a nuclear transport signal, it was covalently coupled with fluorescence labeled bovine serum albumin (BSA-BODIPY) and used with digitonin permeabilized cells as it was shown earlier. In brief, HeLa S6 cells were grown on slides for 24 h in RPMI medium with 10% FCS. Cells were permeabilized for 2 min with 40 μM digitonin in transport buffer. After incubation with 600 nM (PKKKRKVG)4BSA-BODIPY and equivalent amounts of Texas Red labeled BSA (control) in complete transport mixture (30 min), cells were fixed in 4% formaldehyde solution and the slides were evaluated under fluorescence microscope. As shown in FIG. 1 the polypeptide (PKKKRKVG)₄ transports the coupled BSA into the nucleus whereas the free BSA stays outside. If mNLS/BSA-BODIPY was used, no signal was seen in the nucleus same as with wheat germ agglutinin.

### EXAMPLE 2

### Transfection Efficiency of (PKKKRKVG)₄/DNA Complexes

The transfection efficiency of (PKKKRKVG)₄/DNA complexes was compared to that of poly-L-lysine 2.9 kD/DNA complexes. For the luciferase assay 1×10⁵ cells per well in a 24-well culture plate were used for each cell line (16HBE14O-, HeLa S6 and Cos7). Cells were seeded 24 hours before transfection. Depending on the cell line cells reached 30-60% confluence during 24 hours. Before transfection cells were washed with 1 ml of its supplement medium without FCS. The transfections were done in fresh medium in the presence or absence of 10% FCS. The desired amounts of DNA (0.1-3 μg) and (PKKKRKVG)₄, poly-L-Lysine 2.9 kD, polyethylenimine (PEI) 25 kD or fractured Dendrimer were diluted in HBS, 0.15M NaCl or 5% glucose. After mixing each component the vectors were added to the DNA-containing solutions, vortex-mixed gently, and incubated at room temperature for 20 min. The complexes were then added to the cells and incubated for 2 hours at 37° C. and 5% CO₂, at which time the transfection medium was replaced with 1 ml of fresh growth medium containing 10% FCS. Cells were cultured for 24 hours and tested for luciferase gene expression. Cells were lysed with 200 μl lysis buffer per well (Neutral buffered lysis buffer, SIGMA). 10 μl were measured for 10 sec in a luminometer (Lumat LB 9507, BERTHOLD, Germany). FIG. 2 shows that (PKKKRKVG)₄ led to a 100-fold increase of relative light units (RLU) per mg cell protein in comparison to poly-L-lysine. The optimal N/P ratio was around 8 (1 μg CMVL-W; 4.8 μg (PKKKRKVG)₄). Transfection with mNLS/DNA complexes resulted in significantly lower gene transfer efficiency (FIG. 3).

### EXAMPLE 3

### Comparison of Gene Transfer Efficiency of (PKKKRKVG)₄ with Different Non-Viral Vectors

There have been many approaches seeking to increase non-viral gene transfer. In vitro transfection studies have shown that polyfection with fractured Dendrimer or polyethylenimine 25 kD results in high transfection rates. In order to determine the gene delivery efficiency of (PKKKRKVG)₄ a standard Luciferase assay was used. The same transfection protocol as mentioned above was followed. 1 μg of DNA (CMVL-W) per well was complexed with 5 μg poly-L-lysine 2.9 kD (N/P 8), 4.8 μg (PKKKRKVG)₄ (N/P 8), 1.96 μg Polyethylenimine 25 kD PEI (N/P 5) or 3.96 μg fractured Dendrimer (N/P 4.5). FIG. 4 summarizes the results for the transfection efficiencies with different non-viral gene vectors. (PKKKRKVG)₄-mediated gene transfer showed almost as high transfection rates as with Dendrimer/DNA complexes.

### EXAMPLE 4

### Improvement of (PKKKRKVG)₄-Mediated Gene Delivery with Influenza Peptide

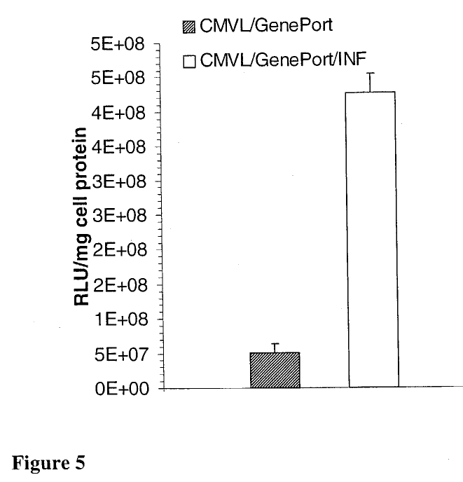

Furthermore, the influence of endosomolytic agents on in vitro gene delivery with DNA (CMVL)/(PKKKRKVG)₄-complexes was investigated. 1 μg CMVL-W was mixed with 4.8 μg (PKKKRKVG)₄ (N/P 8). After incubation for 20 min at room temperature (RT) influenza peptide was added to the complexes (0.78 μg in 5 mM glucose solution) and again incubated for 20 min at RT. Using (PKKKRKVG)₄ with Influenza peptide (INF7a) cell transfection could be enhanced 10-fold compared to studies without endosomolytic agents and obtained cell transfection as high as with DNA/Dendrimer-complexes' or 1000-fold higher as with DNA/poly-L-lysine 2.9 kD/INF7a complexes (FIG. 5 and FIG. 6).

### EXAMPLE 5

### Enhancement of Polyfection with (PKKKRKVG)₄

The next step was to show that (PKKKRKVG)₄ is able to mediate nuclear entrance of DNA/Polymer complexes. Using the above-described standard cell transfection protocol we produced PEI/CMVL-W and Dendrimer/CMVL-W complexes. (PKKKRKVG)₄ was added to the complexes in increasing concentrations and incubated for 20 min at RT. The presence of (PKKKRKVG)₄ resulted in a significant increase of transfection efficiency (FIG. 7). The largest increase of RLUs was seen with 100% confluent cells, were 10-fold higher transfection results were obtained in comparison to transfections without (PKKKRKVG)₄ (FIG. 8).

### EXAMPLE 6

### Stability of (PKKKRKVG)4/DNA Complexes

The stability of vector/DNA complexes was examined by DNase I digestion followed by agarose gel electrophoresis. 3 μg DNA (pEGFP) were complexed with 14.4 μg (PKKKRKVG)₄ (NIP 8), 12 μg fractured Dendrimer (N/P 4.5) or fractured Dendrimer (N/P 4.5) plus 1 μg (PKKKRKVG)₄. The complexes were incubated with 2.5, 5 and IOU DNase I for 1 h at 37° C. After phenol/chloroform extraction, the DNA was precipitated with ethanol over night, air dried and the pellet was resolved in aqua bidest. The DNA was put on a 0.8% agarose gel, which contained lug/ml ethidium bromide. There is a clear difference in DNA protection between Dendrimer/DNA and (PKKKRKVG)₄/DNA complexes. At higher enzyme concentrations the Dendrimer complexed DNA is totally digested whereas the DNA, which was complexed with (PKKKRKVG)₄ still can bee seen. This indicates that (PKKKRKVG)₄ forms highly protected particles with DNA (FIG. 9). In conclusion (PKKKRKVG)₄ forms stable complexes with DNA, which are very resistant against DNase I digestion. In order to demonstrate what happens to the complexes on their way to the cell nucleus we covalently coupled (PKKKRKVG)₄ with fluorescein (FITC, green fluorescence) and DNA was labeled with TOTO-3 (red fluorescence). Using the above-described standard protocol 16HBE14o cells (100% confluent) were transfected with 5 μg DNA (CMVL-W) and 24 μg (PKKKRKVG)₄ (N/P 8) in four chamber culture slides (FALCON #354104). The transfections were stopped after 4, 30 and 52 h by fixation in 4% formaldehyde. As the fluorescence images show most of the complexes were outside the nucleus after 4 h. After 30 h, both (PKKKRKVG)₄ and DNA could be found in the nucleus indicating that the complexes are stable on their way through the cytosol into the nucleus (FIG. 10).

### EXAMPLE 7

### Transfection of HepG2 Cells (ATCC #HB-8065) Using Various Formulations of (PKKKRKVG)₄- and (PKTKRKVG)₄/DNA Complexes

Cells were cultivated in DMEM supplemented with 10% FCS, 100 units/ml penicillin, 100 μg/ml streptomycin and 2 mM glutamine at 37° C. in an athmosphäre of 5% CO₂. The evening preceeding the transfection cells were trypsinized and seed in 96-well culture plates at a density of 50.000 cells per well in 200 pi medium. Immediately before addition of DNA complexes the medium was replaced with 100 μl of fresh medium.

Preparation of DNA Complexes:

Aliquots of 108 μl of 20.2 μM, 40.4 μM, 60.56 μM und 80.7 μM solutions of (PKKKRKVG)₄- and (PKTKRKVG)₄, respectively, in 20 mM HEPES pH 7.4 were transferred to wells A1 to A4 and E1 to E4, respectively, of a U bottom 96-well plate (TPP, Switzerland). Wells B1-D4 and F1-H4 were filled with 180 μl 5% Glucose in 20 mM HEPES pH 7.4.

To wells A1 to A4 und E1 to E4 (containing the peptide solutions) 108 μl each of a DNA stock solution (pCMVLuc; 120 μg DNA (pCMVLuc) in 1800 μl 20 mM HEPES pH 7.4) were added and mixed by pipetting, resulting in polyplexes with charge rations of 2, 4, 6 und 8. After 15 min 108 μl each of an INF7 stock solution (242 μM in 20 mM HEPES pH 7.4) were added to wells A1 to A4 and E1 to E4, respectively, followed by mixing. This corresponds to 6 charge equivalents of INF7 (GLFEAIEGFIENGWEGMIDGWYGC, SEQ ID NO: 19; Plank et al. 1994, loc cit) relative to the amount of DNA. After further 15 min 36 μl each of a 50% glucose solution in water were added to A1 to A4 and E1 to E4, respectively. Subsequently, 180 μl each were transferred from row A to row B and from row E to row F, followed by mixing, then 180 μl were transferred from row B to row C and from row F to row G and so on.

Transfections and Luciferase Assay:

Aliquots of 50 μl each of the resulting dilution series, containing 1, 0.5, 0.25 und 0.125 μg, respectively, of DNA were added in triplicates to the cells in the 96-well culture plate. After 24 hrs the medium was removed, followed by washing with 200 pi PBS per well. One hundred pi of lysis buffer (250 mM Tris pH 7.8; 0.1% Triton X-100) were added per well. After 15 min incubation at room temperature the lysates were mixed once using a multichannel pipettor. Aliquots of 50 pi were transferred to an opaque 96-well plate (Costar) for the luciferase assay followed by addition of 100 μl each of luciferin substrate buffer (60 mM Dithiothreitol, 10 mM Magnesiumsulfat, 1 mM ATP, 30 μM D (−)-Luciferin, in 25 mM Glycyl-Glycin-Puffer pH 7.8). Bioluminescence was recorded and integrated of 12 seconds using a Microplate Scintillation & Luminescence counter ,,Top Count” (Canberra-Packard, Dreieich). Background luminescence was subtracted automatically. Gene expression in ng luciferase per mg protein was calculated according to a calibration curve acquired using a dilution series of 100, 50, 25, 12.6, 6.25, 3.13, 1.57, 0.78, 0.39, 0.2, 0.1, 0.05, 0.025, 0.013, 0.007 und 0 ng of luciferase (Boehringer Mannheim) each in 10 μl lysis buffer each under the same conditions applied for the cell extracts. The protein concentration in cell extracts was determined using the BioRad protein assay adapted for use in a 96-well plate format and using a microtiter plate reader (,,Biolumin 690”, Molecular Dynamics, USA). Protein content was calculated according to a calibration curve acquired with a dilution series of BSA in lysis buffer with BSA concentrations of 50, 33.3, 22.3, 15, 9.9, 6.6, 4.4, 2.9, 2.0, 1.3, 0.9 und 0 ng BSA/μl.

### EXAMPLE 8

### Surface Charge of (PKKKRKVG)₄C-DNA Complexes as Determined by ζ-Potential Measurements

(PKKKRKVG)₄C-DNA complexes were prepared at charge ratios 0.5, 1, 1.2, 1.5, 2, 4, 6, 8 in 20 mM HEPES pH 7.4 by adding 20 μg of DNA (pCMVLuc) in 500 μl buffer to the appropriate amounts of peptide also in 500 μl buffer and mixing. The required amount of peptide is calculated according to

$$Peptide ( μl ) = \frac{DNA ( μg )}{330} \times \frac{CR}{20 \times {c}_{peptide} ( mM )}$$

where CR is the desired charge ratio and c_{peptide} is the concentration of the peptide stock solution determined photometrically. Zeta potentials of the were determined using a Malvern Zetamaster 3000 instrument with refractive index, viscosity and dielectric constant parameters set to those of water as an approximation. FIG. 12 shows that under the experimental conditions DNA can associate the cationic peptide only up to a charge ratio of 2. Above this charge ratio the zeta potential remains constant.

### EXAMPLE 9

### Determination of DNA Compaction by (PKKKRKVG)₄C

Compaction of DNA was assessed by the self-quenching of fluorescein-labeled peptide upon DNA addition. Purified peptide in free thiol form was reacted with fluorescein maleimide (Molecular Probes) and repurified by reverse phase HPLC (Vydac 218TP1022 C-18 column, flow rate 25 ml/min, 0.1% trifluoroacetic acid, 0-40% acetonitrile in 24 min, 40-100% acetonitrile in 5 min, 100% acetonitrile in 5 min). The product peak was lyophilized and redissolved in water. The peptide concentration was determined by a ninhydrin assay (Sarin et al., Anal. Biochem. 117 (1981): 147-157). The linear range of fluorescence versus concentration was determined prior to performing the quenching assay. A DNA amount of 59.4 μg in a volume of 300 μl 20 mM HEPES pH 7.4 was added to well Al of a white, non-transparent 96-well plate (Costar). All other wells contained 50 μl of 20 mM HEPES pH 7.4. Two hundred fifty μl were transferred from A1 to A2, form A2 to A3 and so on. To the resulting dilution series 50 μl of HEPES buffer were added each followed by addition of 100 pi of a 1.5 μM peptide stock solution in 20 mM HEPES pH 7.4. Fluorescence was measured using a Biolumin 690 well plate reader (Molecular Dynamics, USA) with the excitation filter set to 485 nm and the emission filter set to 515 nm. The same experiment was repeated with all components dissolved in 20 mM HEPES pH 7.4/150 mM sodium chloride. Relative fluorescence was calculated according to

$$rel . fluoresc . = \frac{( {F}_{sample} - {F}_{blank} )}{( {F}_{100 \%} - {F}_{blank} )}$$

where F_{blank} is the background fluorescence of 200 μl 20 mM HEPES pH 7.4 and F₁₀₀ is the fluorescence of 200 μl 0.75 μM peptide in the same buffer. FIG. 13 shows the quenching curves obtained and demonstrates that optimal DNA compaction is achieved at a charge ratio of 1:1 in salt-free buffer and at a slightly lower charge ratio in salt-containing buffer.

### EXAMPLE 10

### (PKKKRKVG)4 is a Nuclear Transporter

As the nuclear transport of covalently bound albumin with (PKKKRKVG)₄ was successful, it was further investigated whether (PKKKRKVG)₄ with electrostatic binding to DNA would also function in nuclear transport. A fluorescence in situ hybridisation was performed with a DNA probe against the reporter plasmid pEGFP (=enhanced green fluorescence protein). pEGFP was chosen as the reporter plasmid because it functioned as an internal control to evaluate the specificity of the probe during probe design. Hela S6 cells were transfected with pEGFP/(PKKKRKVG)₄ complexes, pEGFP/(PKTKRKVG)₄ control complexes or naked pEGFP. Transfections were stopped at 2 and 24 hours. The 2 hour time point was chosen as the earliest point of observed localization of plasmid DNA in the nuclear region. Images were taken by fluorescence microscopy and by confocal laser scanning microscopy (CLSM). At 2 hours, fluorescence microscopy shows (FIGS. 14A-F) that only after transfection with DNA/(PKKKRKVG)₄ complexes plasmid DNA could be detected within the nuclear region (FIG. 14D). Whereas transfection with naked DNA or with DNA complexed with the control peptide nuclear localization of plasmid DNA was not seen. At 24 hours (FIG. 14G-L) 68.9% (1,330 of 1,930 cells) of the cells transfected with DNA/(PKKKRKVG)₄ complexes show distinct signals in the nuclear region. Interestingly 14.7% (250 of 1,700 cells) of the cells transfected with DNA/(PKTKRKVG)₄ complexes also show a signal in the nuclear region (FIG. 14K). After transfection with naked plasmid DNA a nuclear signal was not seen.

With the intent to better determine the location of plasmid DNA within the cell nucleus, the same FISH slides were examined by CLSM. Cells were randomly selected and a series of 25 to 35 images with a separation of 250 nm was generated. The mid-nuclear sections were used to detect the distribution of plasmid DNA. FIG. 15 shows the single light optical sections. The images confirm the observation that there is already plasmid DNA in the nucleus at 2 hours after transfection with DNA/(PKKKRKVG)₄ complexes (FIG. 15D). At 24 hours Plasmid DNA complexed with (PKKKRKVG)₄ is accumulated in the nucleus (FIG. 15J). Whereas the DNA complexed with (PKTKRKVG)₄ is arranged around the nuclear membrane at 2 and 24 hours. With naked plasmid DNA there was rarely detected a signal at all.

### EXAMPLE 11

### Evaluation of the Proportion of Transgene Expressing Cells

It was examined how many cells express the transgene product. 16HBE14o-cells were transfected with 1 μg pEGFP/(PKKKRKVG)₄ complexes N/P 8 (24 hours) and measured by flow cytometry. Approximately 50% of the cells showed a GFP (Green Fluorescence Protein) signal after transfecting with (PKKKRKVG)₄ (FIG. 16).

### EXAMPLE 12

### Inhibition of Gene Transfer

The NLS of the SV40 large T-antigen mediates nuclear transport over the classical pathway by importin a and importin β (Gorlich, D. & Kutay, U. Transport between the cell nucleus and the cytoplasm. Annu rev cell dev biol 15, 607-660 (1999)). It was investigated whether DNA/(PKKKRKVG)₄ complexes are transported into the nucleus by the same mechanism. The aim was to inhibit the nuclear uptake of DNA/(PKKKRKVG)₄ complexes through a saturation of the importin mediated transport mechanism through an excess of free (PKKKRKVG)₄. Following a nuclear transport inhibition protocol of Sebestyen (Sebestyen, M. G. et al. DNA vector chemistry: the covalent attachment of signal peptides to plasmid DNA. Nat Biotechnol 16, 80-85 (1998)) a 30 fold molar excess of free (PKKKRKVG)₄ was added to the cells at 0 min, 20 min, and 45 min before the transfection complexes were added to the cells. A complete blockade of gene transfer was found when adding free (PKKKRKVG)₄20 min before the DNA/(PKKKRKVG)₄ complexes (FIG. 17).

### EXAMPLE 13

### Transfection Efficiency of C(YGRKKRRQRRRG)₂₋₄/DNA Complexes

The transfection efficiency of C(YGRKKRRQRRRG)₂₋₄/DNA complexes was examined. For the luciferase assay 3×10⁴ COS7 cells were seeded per well in a 24-well culture plate 24 hours before transfection. Cells reached 60-70% confluence during 24 hours. Before transfection cells were washed with 1 ml of its supplement medium without FCS. The transfections were done in fresh medium in the absence of 10% FCS. The desired amounts of DNA (1 μg) and C(YGRKKRRQRRRG)₂₄ were diluted in HBS. After mixing each component the DNA was vector-containing solutions, mixed gently, and incubated at room temperature for 20 min. The complexes were then added to the cells and incubated for 4 hours at 37° C. and 5% CO₂, at which time the transfection medium was replaced with 1 ml of fresh growth medium containing 10% FCS. Cells were cultured for 24 hours and tested for luciferase gene expression. Cells were lysed with 200 μl lysis buffer per well (Neutral buffered lysis buffer, SIGMA). 10 μl were measured for 10 sec in a luminometer (Lumat LB 9507, BERTHOLD, Germany). FIG. 18 shows that C(YGRKKRRQRRRG)₃ led to significantly higher luciferase gene expression in comparison to C(YGRKKRRQRRRG)₂ and C(YGRKKRRQRRRG)₄. The optimal N/P ratio was around 10.

### EXAMPLE 14

### Transfection Efficiency of C(YCGKKRRQRRRG)₂₋₄/DNA Complexes Compared with Polyethylenimine and Fractured Dendrimers at 4° C. and 37° C.

At low temperatures such as 4° C. energy-dependent processes like the endosomal uptake are suppressed. For this reason transfection efficiency at 4° C. could be an indicator for the extend of endosomal uptake which takes place during the transfection period. Transfection was performed in the way as described above but for the 4° C. experiment the 24 well plate was incubated in a cool room at 4° C. for 4 h. FIG. 19a) shows a decrease of 100-and 130-fold for PEI and fractured Dendrimers when transfections were performed at 4° C. compared to transfection efficiency at 37° C. In contrast, transfection efficiencies of C(YGRKKRRQRRRG)₂, C(YGRKKRRQRRRG)₃; and C(YGRKKRRQRRRG)₄ at 4° C. only decreased 19-, 90-, and 29-fold when compared to transfection efficiency at 37° C.

### EXAMPLE 15

### Effect of C(YGRKKRRQRRRG)₂₋₄ on Gene Transfer Mediated by PEI, Fractured Dendrimers and Lipofectamine

There have been many approaches seeking to increase non-viral gene transfer. In vitro transfection studies have shown that lipofection with Lipofectamine or polyfection with fractured Dendrimers or polyethylenimine 25 kD (PEI) results in high transfection rates. In order to determine the effect of C(YGRKKRRQRRRG)₂₋₄ on gene delivery efficiency of Lipofectamine, fractured Dendrimers or polyethylenimine 25 kD (PEI) a standard Luciferase assay was used. The same transfection protocol as mentioned above was followed. 1 μg of DNA (CMVL-W) per well was complexed with C(YGRKKRRQRRRG)₂₋₄ or poly-L-argine (average number of arginines=41) at N/P=1 and incubated for 10 min. at ambient temperature. Then Polyethylenimine 25 kD PEI (N/P 10), fractured Dendrimer (NIP 4.5) or Lipofectamine (w/w 1/10) was added and complexes further incubated for 10 min. at ambient temperature. FIG. 20 summarizes the results for the transfection efficiencies with different non-viral gene vectors. C(YGRKKRRQRRRG)₂ enhances gene transfer mediated by PEI and fractured dendrimers and C(YGRKKRRQRRRG)₄ enhances gene transfer mediated by Lipofectamine.

### EXAMPLE 16

### Determination of DNA Compactation by C(YGRKKRRQRRRG)₂₋₄

Compactation of DNA was assessed by the quenching of TOTO-1 labeled DNA upon the addition of peptide. Peptides were diluted to concentrations resulting in the indicated NIP ratios with HBS to a volume of 100 μl. 100 μl of a solution containing 0.25 μg of TOTO-1 labeled DNA (HBS, every 20 base pair labeled) was added to the peptides. Fluorescence was measured in 96-well plates and values reported refer to the percentage of fluorescence when fluorescence of labeled DNA was measured without the addition of peptide. FIG. 21 demonstrates that DNA compaction is achieved at an N/P ratio of 10 for all of the peptides and that the degree of DNA compaction depends on the molecular weight of the peptides.

1. A polypeptide comprising at least two peptide monomers, wherein each peptide monomer comprises an amino acid sequence which serves as a nuclear localization sequence or an amino acid sequence which serves as a protein transduction domain in eukaryotic cells.

2. The polypeptide of claim 1, wherein the nuclear localization sequence comprises an amino acid sequence selected from the group consisting of
(SEQ ID NO:1)

| | |
|---|---|
| (a) | PKKKRKV; |

(SEQ ID NO:5)

| | |
|---|---|
| (b) | PKKKRKVG; |

(SEQ ID NO:2)

| | |
|---|---|
| (c) | NLSKRPAAIKKAGQAKKKK; |

(SEQ ID NO:4)

| | |
|---|---|
| (d) | NQSSNFGPMKGGNFGGRSSGPYGGGGQYFAKPRNQGGY; |
| | and |

(SEQ ID NO:3)

| | |
|---|---|
| (e) | PAAKRVKLD. |

3. The polypeptide of claim 1, wherein the protein transduction domain comprises an amino acid sequence selected from the group consisting of

| | | |
|---|---|---|
| (a) | YGRKKRRQRRR; | (SEQ ID NO:20) |
| (b) | KRIHPRLTRSIR; | (SEQ ID NO:22) |
| (c) | PPRLRKRRQLNM; | (SEQ ID NO:23) |
| (d) | RRQRRTSKLMKR; | (SEQ ID NO:24) |
| (e) | RQIKIWFQNRRMKWKK | (SEQ ID NO:21) |
| (f) | KLALKLALKALKAALKLA | (SEQ ID NO:29) |
| (g) | GWTLNSAGYLLGKINLKALAALAKKIL | (SEQ ID NO:25) |
| (h) | AAVALLPAVLLALLAP | (SEQ ID NO:26) |
| (I) | AAVLLPVLLAAP, | (SEQ ID NO:27) |
| | and | |
| (j) | VTVLALGALAGVGVG. | (SEQ ID NO:28) |

4. The polypeptide of any one of claims 1 to 3, wherein at least one monomer comprises a nuclear localization sequence and at least one monomer comprises a protein transduction domain.

5. The polypeptide of any one of claims 1 to 4, wherein the polypeptide comprises at least two monomers comprising a nuclear localization sequence and wherein the nuclear localization sequences in the different monomers are the same.

6. The polypeptide of any one of claims 1 to 4, wherein the polypeptide comprises at least two monomers comprising a nuclear localization signal and wherein the nuclear localization sequences in the different monomers are of different types.

7. The polypeptide of any one of claims 1 to 6, wherein the polypeptide comprises at least two monomers comprising a protein transduction domain and wherein the protein transduction domains in the different monomers are the same.

8. The polypeptide of any one of claims 1 to 6, wherein the polypeptide comprises at least two monomers comprising a protein transduction domain and wherein the protein transduction domains in the different monomers are of different types.

9. The polypeptide of any one of claims 1 to 8 comprising at least 3 peptide monomers.

10. The polypeptide of claim 9 comprising at least 4 polypeptide monomers.

11. The polypeptide of claim 5, which is the tetramer (PKKKRKV)₄ or the tetramer (PKKKRKVG)₄.

12. The polypeptide of claim 7, which is the dimer, trimer or tetramer C(YGRKKRRWRRRG)₂₋₄.

13. A polypeptide conjugate comprising at least two of the polypeptides of any one of claims 1 to 12, which are covalently linked to each other.

14. The polypeptide conjugate of claim 13, wherein the covalent linkage is an amid, disulfid, ester, ether, sulfonamid or thiol bond, a carbon-nitrogen double bond or a carbon-nitrogen single bond.

15. The polypeptide of any one of claims 1 to 12 or the polypeptide conjugate of claim 13 or 14, which is modified by covalent linkage to another molecule.

16. The polypeptide or polypeptide conjugate of claim 15, wherein the molecule is a ligand binding to a receptor or a signal enhancing gene transfer into eukaryotic cells.

17. A complex comprising at least one polypeptide of any one of claims 1 to 12, 15 or 16 and/or at least one polypeptide conjugate of any one of claims 13 to 16 and at least one molecule.

18. The complex of claim 17, wherein the polypeptide or polypeptide conjugate and the molecule interact by ionic bonds.

19. The complex of claim 17 or 18, which is furthermore linked to another molecule.

20. The complex of claim 19, wherein the molecule is an endosomolytic agent.

21. The complex of claim 19, wherein the molecule is polyethylenglycol.

22. A process for preparing the complex of any one of claims 17 to 21 comprising the step of contacting the polypeptide and/or polypeptide conjugate and the molecule under conditions which allow the formation of the complex.

23. A pharmaceutical composition comprising the polypeptide of any one of claims 1 to 12, 15 or 16, a polypeptide conjugate of any one of claims 13 to 16 and/or a complex of any one of claims 17 to 21 and, optionally, a pharmaceutically acceptable carrier.

24. A process for transferring a molecule into eukaryotic cells comprising the step of contacting the cells with
(i) the polypeptide of any one of claims 1 to 12, 15 or 16 and/or the polypeptide conjugate of any one of claims 13 to 16 in the presence of the molecule; and/or
(ii) the complex of any one of the claims 17 to 21; and/or
(iii) the pharmaceutical composition of claim 23.

25. A kit comprising the polypeptide of any one of claims 1 to 12, 15 or 16, the polypeptide conjugate of any one of claims 13 to 16 or the complex of any one of claims 17 to 21.

26. Use of a polypeptide of any one of claims 1 to 12, 15 or 16, the polypeptide conjugate of any one of claims 13 to 16 or the complex of any one of claims 17 to 21 for transferring molecules into eukaryotic cells.

27. The complex of claim 17 or the use of claim 26, wherein the molecule is a negatively charged molecule.

28. The complex or use of claim 27, wherein the negatively charged molecule is a nucleic acid molecule.


SEQUENCE LISTING (32 sequences)

SEQ ID NO: 1 - protein, 7 aa, Artificial Sequence
  Description of Artificial Sequence SV40 virus large T-antigen minimal nuclear localization sequence
PKKKRKV

SEQ ID NO: 2 - protein, 16 aa, Artificial Sequence
  Description of Artificial Sequence nucleoplasmin bipartite nuclear localization sequence
KRPAAIKKAGQAKKKK

SEQ ID NO: 3 - protein, 9 aa, Artificial Sequence
  Description of Artificial Sequence c-myct nuclear localization sequence
PAAKRVKLD

SEQ ID NO: 4 - protein, 38 aa, Artificial Sequence
  Description of Artificial Sequence hRNPA1 M9 nuclear localization sequence
NQSSNFGPMKGGNFGGRSSGPYGGGGQYFAKPRNQGGY

SEQ ID NO: 5 - protein, 8 aa, Artificial Sequence
  Description of Artificial Sequence synthetic peptide based on the SV40 virus large T antigen wild-type nuclear localization sequence
PKKKRKVG

SEQ ID NO: 6 - protein, 8 aa, Artificial Sequence
  Description of Artificial Sequence synthetic peptide based on the SV40 virus large T antigen mutant nuclear localization sequence
PKTKRKVG

SEQ ID NO: 7 - protein, 11 aa, Artificial Sequence
  Description of Artificial Sequence c-myct nuclear localization sequence
RQRRNELKRSF

SEQ ID NO: 8 - protein, 41 aa, Artificial Sequence
  Description of Artificial Sequence importin- alpha nuclear localization sequence from the IBB domain
RMRKFKNKGKDTAELRRRRVEVSVELRKAKKDEQILKRRNV

SEQ ID NO: 9 - protein, 8 aa, Artificial Sequence
  Description of Artificial Sequence myoma T protein nuclear localization sequence
VSRKRPRP

SEQ ID NO: 10 - protein, 8 aa, Artificial Sequence
  Description of Artificial Sequence myoma T protein nuclear localization sequence
PPKKARED

SEQ ID NO: 11 - protein, 8 aa, Artificial Sequence
  Description of Artificial Sequence human p53 nuclear localization sequence
PQPKKKPL

SEQ ID NO: 12 - protein, 12 aa, Artificial Sequence
  Description of Artificial Sequence murine c-abl IV nuclear localization sequence
SALIKKKKKMAP

SEQ ID NO: 13 - protein, 5 aa, Artificial Sequence
  Description of Artificial Sequence influenza virus NS1 nuclear localization sequence
DRLRR

SEQ ID NO: 14 - protein, 7 aa, Artificial Sequence
  Description of Artificial Sequence influenza virus NS1 nuclear localization sequence
PKQKKRK

SEQ ID NO: 15 - protein, 10 aa, Artificial Sequence
  Description of Artificial Sequence hepatitis virus delta antigen nuclear localization sequence
RKLKKKIKKL

SEQ ID NO: 16 - protein, 10 aa, Artificial Sequence
  Description of Artificial Sequence murine Mx1 nuclear localization sequence
REKKKFLKRR

SEQ ID NO: 17 - protein, 20 aa, Artificial Sequence
  Description of Artificial Sequence human poly (ADP-ribose) polymerase bipartite nuclear localization sequence
KRKGDEVDGVDEVAKKKSKK

SEQ ID NO: 18 - protein, 17 aa, Artificial Sequence
  Description of Artificial Sequence human glucocorticoid receptor bipartite nuclear localization sequence
RKCLQAGMNLEARKTKK

SEQ ID NO: 19 - protein, 24 aa, Artificial Sequence
  Description of Artificial Sequence influenza virus 7 protein transport domain sequence
GLFEAIEGFIENGWEGMIDGWYGC

SEQ ID NO: 20 - protein, 11 aa, Artificial Sequence
  Description of Artificial Sequence HIV-1 TAT protein basic protein transduction domain sequence
YGRKKRRQRRR

SEQ ID NO: 21 - protein, 16 aa, Artificial Sequence
  Description of Artificial Sequence Drosophila basic protein transduction domain from the Antennapedia third helix
RQIKIWFQNRRMKWKK

SEQ ID NO: 22 - protein, 12 aa, Artificial Sequence
  Description of Artificial Sequence synthetic peptide of a basic protein transduction domain
KRIHPRLTRSIR

SEQ ID NO: 23 - protein, 12 aa, Artificial Sequence
  Description of Artificial Sequence synthetic peptide of a basic protein transduction domain
PPRLRKRRQLNM

SEQ ID NO: 24 - protein, 12 aa, Artificial Sequence
  Description of Artificial Sequence synthetic peptide of basic protein transduction domain
RRQRRTSKLMKR

SEQ ID NO: 25 - protein, 27 aa, Artificial Sequence
  Description of Artificial Sequence transportan hydrophobic protein transduction domain
GWTLNSAGYLLGKINLKALAALAKKIL

SEQ ID NO: 26 - protein, 16 aa, Artificial Sequence
  Description of Artificial Sequence transportan hydrophobic protein transduction domain
AAVALLPAVLLALLAP

SEQ ID NO: 27 - protein, 12 aa, Artificial Sequence
  Description of Artificial Sequence transportan hydrophobic protein transduction domain
AAVLLPVLLAAP

SEQ ID NO: 28 - protein, 15 aa, Artificial Sequence
  Description of Artificial Sequence transportan hydrophobic protein transduction domain
VTVLALGALAGVGVG

SEQ ID NO: 29 - protein, 18 aa, Artificial Sequence
  Description of Artificial Sequence synthetic sequence of a hydrophobic protein transduction domain
KLALKLALKALKAALKLA

SEQ ID NO: 30 - protein, 13 aa, Artificial Sequence
  Description of Artificial Sequence synthetic peptide of a nuclear localization sequence
CYGRKKRRQRRRG

SEQ ID NO: 31 - protein, 25 aa, Artificial Sequence
  Description of Artificial Sequence synthetic peptide of a nuclear localization sequence
CYGRKKRRQRRRGYGRKKRRQRRRG

SEQ ID NO: 32 - protein, 37 aa, Artificial Sequence
  Description of Artificial Sequence synthetic peptide of a nuclear localization sequence
CYGRKKRRQRRRGYGRKKRRQRRRGYGRKKRRQRRRG